(19) United States
(12) Patent Application Publication
(10) Pub. No.: US 2024/0228979 A1
(43) Pub. Date: Jul. 11, 2024

(54) NOVEL TOMATO YELLOW LEAF CURL VIRUS ISOLATE AND INFECTIOUS CLONES THEREOF

(71) Applicant: Agricultural Company Corporation Daenong seed Co., Ltd., Jeollanam-do (KR)

(72) Inventors: Suk Chan LEE, Gyeonggi-do (KR); Nattanong BUPI, Gyeonggi-do (KR); Nam Ju AHN, Jeollanam-do (KR); Woo Seong AHN, Jeollanam-do (KR); Dong Hyeon KIM, Gyeonggi-do (KR); Hyojin IM, Chungcheongnam-do (KR)

(21) Appl. No.: 18/400,143

(22) Filed: Dec. 29, 2023

(30) Foreign Application Priority Data
Jan. 6, 2023 (KR) 10-2023-0002530
Feb. 3, 2023 (KR) 10-2023-0015207
Feb. 3, 2023 (KR) 10-2023-0015208
Feb. 3, 2023 (KR) 10-2023-0015209

(51) Int. Cl. C12N 7/00; C12N 15/82; C12Q 1/70

(52) U.S. Cl. CPC C12N 7/00; C12N 15/8202; C12Q 1/701; C12N 2750/12021

(57) ABSTRACT

Disclosed are a novel Korean isolate of tomato yellow leaf curl virus (TYLCV) and infectious clones thereof, wherein: the use of an inventive recombinant vector comprising the nucleotide sequence of tomato yellow leaf curl virus (TYLCV) and Agrobacterium tumefaciens comprising the recombinant vector can effectively induce tomato yellow leaf curl virus infection in a crop even without an insect vector; an inventive infection system, compared with existing TYLCV Korean isolates, shows excellent infectivity in a plant, and thus can induce symptoms even in TYLCV-resistant varieties of tomatoes having existing widely used Ty-1 or Ty-2 resistant gene, so that the infectious system can be utilized as an artificial infection system in the future and can be applied in various studies in the future, such as a host plant range for tomato yellow leaf curl virus (TYLCV) and the correlation with the host plant.

### CROSS-REFERENCE TO RELATED APPLICATION(S)

This application is based on and claims priority under 35 U.S.C. 119 to Korean Patent Application No. 10-2023-0002530, filed on Jan. 6, 2023, in the Korean Intellectual Property Office, the disclosure of which is herein incorporated by reference in its entirety.

Also, This application is based on and claims priority under 35 U.S.C. 119 to Korean Patent Application No. 10-2023-0015207, 10-2023-0015208 and 10-2023-0015209, filed on Feb. 3, 2023, in the Korean Intellectual Property Office, the disclosure of which is herein incorporated by reference in its entirety.

This application contains references to amino acid sequences and/or nucleic acid sequences which have been submitted concurrently herewith as the sequence listing XML file entitled “000355 us_SequenceListing.XML”, file size 33,422 bytes, created on 28 Dec. 2023. The aforementioned sequence listing is hereby incorporated by reference in its entirety pursuant to 37 C.F.R. § 1.52(e)(5).

### FIELD

The present disclosure is made with the support of the Ministry of Agriculture, Food and Rural Affairs, Republic of Korea, under Project No. 322066-03-1-CG000. The research project is titled “Digital Breeding Conversion Technology Development” and falls within the research program named “Nurturing new tomato varieties resistant to multiple viruses using digital breeding technology”. This project is carried out by the Agricultural Company Corporation DaeNong Seed Company, under the management of the Korea Institute of Agriculture, Food and Rural Affairs Technology Planning and Evaluation, from Apr. 1, 2022, to Dec. 31, 2024.

The present disclosure relates to a novel tomato yellow leaf curl virus (TYLCV) isolate and infectious clones thereof.

### BACKGROUND

Tomato yellow leaf curl virus (TYLCV) is a virulent pathogen that causes devastating economic losses annually in the cultivation of tomatoes worldwide and was first isolated from tomato crops grown in the southern island region of Korea in 2008. The virus has now spread, and the outbreak of TYLCV has been persistently reported across the country. There has been a TYLCV outbreak in tomato crops every year, which poses a serious problem for tomato production.

In general, tomato plants infected with TYLCV show typical yellow leaf curl disease symptoms, such as leaf curling, leaf yellowing of young leaves, and stunting. In particular, the outbreak of the symptoms immediately after planting seedling makes the harvest of tomatoes almost impossible, and the outbreak of the symptoms during growth causes no influence on fruits but poor fruiting, resulting in a 70-80% reduction in tomato yield and quality degradation.

TYLCV is transmitted by whitefly (Bemisia tabaci Genn.) and may cause an infection in a persistent and circulative manner, and moreover, whitefly has already become indigenous to Korea and thus is difficult to control, and therefore, the infection with TYLCV is very difficult to prevent. Moreover, recent studies have newly revealed that TYLCV can be transmitted through seeds, suggesting the possibility of widespread outbreak of TYLCV into new regions.

However, several TYLCV resistance genes including Ty-1, Ty-2, Ty-3, Ty-4, and Ty-5 were identified in wild-type tomato species, and in recent years, most tomato farmers in Korea effectively protect tomatoes from TYLCV infection by cultivating TYLCV-resistant varieties containing different Ty loci. However, the genetic diversity of TYLCV populations may bring about viruses with new infectivity through various mechanisms that can result in variations in the virus population, such as mutations, inversions of nucleic acid sequences, recombination, and mixed infections, which can lead to the breakdown of resistance and the re-emergence of new TYLCV diseases.

Therefore, it is necessary to identify the presence or absence of new TYLCV that can cause severe symptoms in tomato plants as well as can infect a conventional TYLCV-resistant variety of tomatoes and to identify the infectivity thereof.

### PRIOR ART DOCUMENT

### Patent Document

(Patent Document 01) Korean Patent No. 10-1430805 (8 Aug. 2014)

### SUMMARY

The present inventors have made intensive research efforts to develop infectious clones of a novel tomato yellow leaf curl virus (TYLCV) isolate and an artificial infection system using the same. As a result, the present inventors established an effect of reproducing infectious activity in a plant by introducing a binary vector comprising infectious clones of a novel tomato yellow leaf curl virus isolate into the plant through agro-inoculation, and thus completed the present disclosure.

Accordingly, an aspect of the present disclosure is to provide a nucleic acid molecule comprising the nucleotide sequence of SEQ ID NO: 1, the nucleotide sequence of SEQ ID NO: 2, the nucleotide sequence of SEQ ID NO: 3, or a combination thereof.

Another aspect of the present disclosure is to provide a recombinant vector comprising the nucleic acid molecule.

Still another aspect of the present disclosure is to provide Agrobacterium tumefaciens comprising the recombinant vector.

Still another aspect of the present disclosure is to provide a tomato yellow leaf curl virus (TYLCV)-infected plant including the recombinant vector.

Still another aspect of the present disclosure is to provide a composition for diagnosing tomato yellow leaf curl virus (TYLCV) infection, the composition containing an agent for detecting a nucleic acid molecule comprising the nucleotide sequence of SEQ ID NO: 1, the nucleotide sequence of SEQ ID NO: 2, the nucleotide sequence of SEQ ID NO: 3, or a combination thereof and a fragment thereof.

Still another aspect of the present disclosure is to provide a method for diagnosing TYLCV infection in a plant by using the composition for diagnosing TYLCV infection.

Still another aspect of the present disclosure is to provide a method for inducing TYLCV symptoms in a plant, the method comprising transfecting a plant with the recombinant vector.

The present inventors have made intensive research efforts to develop an artificial infection system for research of tomato yellow leaf curl virus (TYLCV). As a result, the present inventors established an effect of reproducing infectious activity in a plant by introducing a binary vector comprising infectious clones of a novel tomato yellow leaf curl virus (TYLCV) isolate into the plant through agro-inoculation.

In accordance with an aspect of the present disclosure, there is provided a nucleic acid molecule comprising the nucleotide sequence of SEQ ID NO: 1, the nucleotide sequence of SEQ ID NO: 2, the nucleotide sequence of SEQ ID NO: 3, or a combination thereof.

As used herein, the term “tomato yellow leaf curl virus (TYLCV)” refers to a plant virus belonging to the family Geminiviridae and the genus Begomovirus, which is known to occur mainly in tomatoes, but the virus has recently been reported to infect various crops including the Solanaceae family crops, such as chili, paprika, and tobacco, the Cucurbitaceae family crops, and the Leguminosae family crops. An infection with TYLCV causes leaf curling symptoms, wherein the leaves of infected plants curl, turn yellow, and become smaller. On the other hand, TYLCV, first reported in Korea, was identified to have a single genome (Lee et al., 2010). Specifically, TYLCV of the present disclosure consists of a single-stranded circular DNA monopartite genome (DNA-A) of approximately 2.6-2.8 Kb encapsulated in a bi-decahedral shape.

In accordance with another aspect of the present disclosure, there is provided a recombinant vector comprising a nucleic acid molecule.

Specifically, the present disclosure provides a recombinant vector comprising a nucleic acid molecule comprising the nucleotide sequence of SEQ ID NO: 1, the nucleotide sequence of SEQ ID NO: 2, the nucleotide sequence of SEQ ID NO: 3, or a combination thereof.

The nucleic acid molecule comprises a nucleotide sequence derived from a virus that causes a disease in a plant, wherein the virus may be tomato yellow leaf curl virus (TYLCV), and specifically, the virus refers to a new TYLCV that has been reported to occur in Korea, but is not limited thereto.

Therefore, the nucleic acid molecule consists of nucleotide sequences derived from novel TYLCV Korean isolates KG3, KG4, and KG5, wherein the nucleotide sequence of SEQ ID NO: 1 is derived from the TYLCV Korean isolate KG3, the nucleotide sequence of SEQ ID NO: 2 is derived from the TYLCV Korean isolate KG4, and the nucleotide sequence of SEQ ID NO: 3 is derived from the TYLCV Korean isolate KG5.

As used herein, the term “vector” refers to a vehicle as a means for expressing a target gene in a host cell, and this comprises: a plasmid vector; an E. coli vector; an Agrobacterium vector; and a bacterial or viral vector, such as a bacteriophage vector, an adenovirus vectors, a retroviral vector, and an adeno-associated viral vector, and specifically may be E. coli, but is not limited thereto.

In an embodiment of the present disclosure, the vector may be typically constructed as a cloning vector or an expression vector. Specifically, the vector usable in the present disclosure may be constructed by engineering plasmids (e.g., pSK349, pSC101, ColE1, pBR322, pUC8/9, pHC79, pGEX series, pET series, pUC19, etc.), phages (e.g., λgt·λ4B, λ-Charon, λΔz1, M13, etc.), or viruses (e.g., SV40, etc.), which are often used in the art.

In another embodiment of the present disclosure, the vector may be constructed by using a eukaryotic cell or prokaryotic cell as a host cell.

Specifically, the recombinant vector may be a recombinant vector for host cell expression comprising: (a) the TYLCV nucleotide sequence of SEQ ID NO: 1, the TYLCV nucleotide sequence of SEQ ID NO: 2, the TYLCV nucleotide sequence of SEQ ID NO: 3, or a combination thereof; (b) a promoter that is operatively linked to the nucleotide sequence to form an RNA molecule in a host cell; and (c) a poly A signal sequence that acts on the host cell to induce polyadenylation at the 3′-terminus of the RNA molecule.

More specifically, for example, when the vector of the present disclosure is an expression vector and a prokaryotic cell is used as a host, the vector generally includes a strong promoter capable of implementing transcription (e.g., pL\hat{ } promoter, trp promoter, lac promoter, T7 promoter, tac promoter, etc.), a ribosome binding site for initiation of translation, and a transcriptional/translational termination sequence. When E. coli is used as a host cell, the promoter and operator sites on the E. coli tryptophan biosynthesis pathway (Yanofsky, C., J. Bacteriol., 158:1018-1024(1984)) and the leftward promoter from phage λ (pL\hat{ }promoter, Herskowitz, I. and Hagen, D., Ann. Rev. Genet., 14:399-445(1980)) may be used as a control region.

When the vector of the present disclosure is an expression vector and a eukaryotic cell is used as a host, a promoter derived from a genome of a mammalian cell (e.g., a metallothionein promoter) or a promoter derived from a mammalian virus (e.g., adenovirus late promoter, vaccinia virus 7.5 K promoter, SV40 promoter, cytomegalovirus promoter, HSV tk promoter) may be used, and the vector generally has a polyadenylation sequence as a transcription termination sequence.

However, when the vector of the present disclosure is applied to a plant cell, a suitable promoter in the present disclosure may be any promoter that can be typically used in the art for the introduction of a gene into a plant, and examples thereof may include maize-ubiquitin promoter, cauliflower mosaic virus (CaMV) 35S promoter, nopaline synthase (nos) promoter, Figwort mosaic virus 35S promoter, sugarcane bacilliform virus promoter, commelina yellow mottle virus promoter, photo-inducible promoter of small subunit of ribulose-1,5-bis-phosphate carboxylase (ssRUBISCO), rice cytosolic triosphosphate isomerase (TPI) promoter, adenine phosphoribosyltransferase (APRT), octopine synthase promoter, blue copper binding protein (BCB) promoter, SP6 promoter, T7 promoter, T3 promoter, and PM promoter, but are not limited thereto.

As used herein, the term “operatively linked” refers to functional linking between a nucleic acid expression control sequence (e.g., a promoter, a signal sequence, or an array of transcription factor binding sites) and another nucleic acid sequence, whereby the control sequence controls the transcription and/or translation of the another nucleic acid sequence.

The poly A signal sequence inducing polyadenylation at the 3′-end, which is suitable in the present disclosure, includes those derived from the nopaline synthase gene of Agrobacterium tumefaciens (NOS 3′ end) (Bevan et al., Nucleic Acids Research, 11(2):369-385(1983)), those derived from the octopine synthase gene of Agrobacterium tumefaciens, the 3′-end region of the protease inhibitor I or Il gene from potato or tomato, CaMV 35S terminator sequences, and octopine synthase (OCS) terminator sequences. Most specifically, the 3′-end poly A signal sequence inducing polyadenylation suitable in the present disclosure is a termination sequence (Tnos) of the nopaline synthase gene.

In addition, the vector of the present disclosure may comprise, as a selective marker, an antibiotic-resistant gene that is ordinarily used in the art, and examples thereof may include resistant genes against ampicillin, gentamicin, carbenicillin, chloramphenicol, streptomycin, kanamycin, geneticin, neomycin, and tetracycline.

Optionally, the vector may additionally deliver a gene encoding a reporter molecule (e.g., luciferase and glucuronidase).

The vector system of the present disclosure may be constructed by various methods known in the art, and a specific method thereof is disclosed in the literature (Sambrook, et al., Molecular Cloning, A Laboratory Manual, Cold Spring Harbor Laboratory Press (2001)), which is incorporated herein by reference.

As described above, the recombinant vector of the present invention comprises a nucleic acid molecule comprising a nucleotide sequence derived from a virus that causes a disease in a plant and therefore has the most desirable usability for the plant.

To identify the infectivity of a virus isolate in a plant and the usability thereof as an artificial infectious strain in the future, the expression of the recombinant vector in the plant is essential and a specific example of the recombinant vector for expression in the plant for this purpose may be an Agrobacterium binary vector.

In an example of the present disclosure, recombinant vectors comprising nucleic acid molecules comprising nucleotide sequences derived from the novel TYLCV Korean isolates KG3, KG4, and KG5 were constructed and Agrobacterium transformed with the recombinant vectors was identified to exhibit excellent infectivity in plants, suggesting the usability thereof as artificial infectious clones in the future.

The “Agrobacterium” is one of the motile soil microorganisms living in the soil. Agrobacterium has a tumor-induced (Ti) plasmid and thus causes abnormal proliferation of tissue cells by infecting a plant through wounds.

The “binary vector” refers to a vector divided into two plasmids consisting of a plasmid having a left border (LB) and a right border (RB), which are necessary for mobility in a tumor inducible (Ti) plasmid, and a plasmid containing a gene necessary for transferring a target nucleotide sequence.

The Agrobacterium for transformation of the present disclosure may be any one that is suitable for the expression of the nucleotide sequence of TYLCV of the present disclosure or the infection thereby. In particular, the Agrobacterium strain for plant transformation in the present disclosure is usually Agrobacterium tumefaciens GV3101.

Accordingly, in accordance with still another aspect of the present disclosure, there is provided Agrobacterium tumefaciens comprising the recombinant vector.

In an embodiment of the present disclosure, the Agrobacterium tumefaciens is Accession No. KACC92454P, which was deposited in the Korean Agricultural Culture Collection (KACC) on 21 Sep. 2022.

In an embodiment of the present disclosure, the Agrobacterium tumefaciens is Accession No. KACC92455P, which was deposited in the Korean Agricultural Culture Collection (KACC) on 21 Sep. 2022.

In an embodiment of the present disclosure, the Agrobacterium tumefaciens is Accession No. KACC92456P, which was deposited in the Korean Agricultural Culture Collection (KACC) on 21 Sep. 2022.

The recombinant vector of the present disclosure may be introduced into Agrobacterium by various methods known to a person skilled in the art, and examples of the methods may be particle bombardment, electroporation, transfection, a lithium acetate method, heat shock, and a freezethaw method, but are not limited thereto.

As used herein, the term “transformation” means that exogenous DNA or RNA is absorbed into a cell to change the genotype of the cell. The host cell may include, but not limited to, a plant cell, a prokaryotic cell, a yeast cell, or an insect cell.

It was identified in an example of the present disclosure that plants infected with Agrobacterium tumefaciens transformed with the recombinant vector effectively showed TYLCV-specific symptoms.

Specifically, in an embodiment of the present disclosure, it was verified that Agrobacterium tumefaciens comprising the recombinant vector exhibited excellent infectivity in a TYLCV-sensitive variety of tomatoes, for example, Solanum lycopersicum cv. Money Maker, and also exhibited excellent infectivity in a TYLCV-resistant variety of tomatoes having Ty-1 or Ty-2 resistance genes and caused TYLCV-specific symptoms.

As described above, the recombinant vectors of the present disclosure comprise nucleotide sequences derived from TYLCV-KG3, TYLCV-KG4, and TYLCV-KG5, which are novel TYLCV Korean isolates, and it was identified in an example of the present disclosure that Agrobacterium tumefaciens comprising respective recombinant vectors showed different levels of infectivity and symptoms in plants.

It has been known that existing TYLCV Korean isolates may be classified into TYLCV-KG1 and TYLCV-KG2, with which plants were infected to show different levels of symptoms.

The TYLCV Korean isolate KG1 consists of the nucleotide sequence of TYLCV set forth in SEQ ID NO: 4, which is a sequence corresponding to GenBank Accession No. HM130912 (Lee et al., 2010). This isolate encompasses isolates having a genome of 2,774 nucleotides in nine regions of Gyeongsang-do and Jeolla-do and showed a moderate level of severe symptoms in tomatoes. The TYLCV Korean isolate KG2 consists of the nucleotide sequence of TYLCV set forth in SEQ ID NO: 5, which is a sequence corresponding to GenBank Accession No. HM130913 (Lee et al., 2010). This isolate encompasses isolates having a genome of 2,781 nucleotides in four regions of Jeju-do and Jeolla-do and showed a slight level of symptoms in tomatoes.

However, in an embodiment of the present disclosure, plants infected with TYLCV-KG3- and TYLCV-KG4-derived Agrobacterium tumefaciens are characterized by showing more severe symptoms compared with those infected with the TYLCV Korean isolate TYLCV-KG1.

In an embodiment of the present disclosure, plants infected with TYLCV-KG5-derived Agrobacterium tumefaciens are characterized by showing a slight level of symptoms like in the existing TYLCV Korean isolate TYLCV-KG2.

Specifically, it was identified in an example of the present disclosure that the plants infected with the TYLCV-KG3-derived and TYLCV-KG4-derived Agrobacterium tumefaciens, compared with those infected with TYLCV-KG1-derived Agrobacterium tumefaciens, showed more severe TYLCV-specific symptoms and high symptom severity scores and viral DNA copy numbers. Whereas the plants infected with the TYLCV-KG5-derived Agrobacterium tumefaciens showed a slight level of symptoms, which were identical/similar to a slight level of symptoms induced from TYLCV-KG2.

In another embodiment of the present disclosure, the use of TYLCV-KG3- and TYLCV-KG4-derived Agrobacterium tumefaciens is characterized by showing high infectivity in a TYLCV-resistant variety of tomatoes, for example, a TYLCV-resistant variety of tomatoes into which TYLCV resistance-related Ty-1 or Ty-2 locus has been introduced.

Specifically, it was identified in an example of the present disclosure that in cases of the infection with TYLCV-KG3-derived Agrobacterium tumefaciens, leaf yellowing and curling were persistently observed in all the TYLCV-resistant variety of tomatoes, and in cases of TYLCV-KG4, symptoms were observed in only the TYLCV-resistant variety of tomatoes into which the Ty-1 locus has been introduced. However, specific symptoms were not observed in all the TYLCV-resistant variety of tomatoes inoculated with existing TYLCV-KG1 compared with the Mock treatment group.

So far, research on virus-resistant crops has been conducted by various domestic and foreign institutions and seed companies, wherein novel varieties have been developed through cross-breeding with species having resistance genes to TYLCV, mainly Solanum species, which are tomato relatives (wild species), such as S. peruvianum, S. chilense, S. habrochaites, S. pimpinellifolium, and S. cheesmaniae. Seven genes (Ty-1, Ty-2, Ty-3, Ty-3a, Ty-4, Ty-5, and Ty-6) in wild tomatoes are known as resistance genes to TYLCV so far, and it has been reported that Ty-1, -3, -4, and -6 were derived from S. chilense, Ty-2 was derived from S. habrochaites, and Ty-5 was derived from S. peruvianum. It was also reported that the loci thereof are on chromosome 6 for the Ty-1 gene, chromosome 11 for the Ty-2 gene, chromosome 3 for the Ty-4 gene, chromosome 4 for the Ty-5 gene, and chromosome 10 for the Ty-6 gene. The Ty-3 and Ty-3a are alleles present at the same locus on chromosome 6. The gene markers are actively used to breed TYLCV-resistant lineages and varieties, and several TYLCV-resistant varieties on the market are being cultivated in farms.

In order to discover new resistance genetic resources for TYLCV variants, clones for all TYLCV variants occurring in Korea are needed. In particular, various mutant virus species exist in tomato cultivation packaging, and thus novel TYLCV variant isolates are secured therefrom, so that new genetic resources with TYLCV resistance that can be used for breeding industrial varieties can be discovered.

Therefore, the novel TYLCV isolates of the present disclosure can play an important role in discovering TYLCV-resistant recourses and establishing resistant varieties. Specifically, the use of KG3 and KG4 inducing TYLCV-specific symptoms in existing resistant varieties of tomatoes allows for the expectation of the possibility of discovering novel resistant genes, whereas KG5 inducing a slight level of symptoms, like the existing isolate KG2 revealed in Korea, is an attenuated virus and can be used to induce the resistance to various environmental stresses or prevent highly virulent viruses in advance.

In accordance with still another aspect of the present disclosure, there is provided a TYLCV-infected plant transformed with the recombinant vector.

As used herein, the term “plant” means encompassing all of a mature plant as well as a plant cell, a plant tissue, a plant seed, and the like, which can develop into a mature plant.

The plant of the present disclosure is not particularly limited, and specifically, tomatoes may be preferable. The plant may include crops, such as the families Leguminosae, Cucurbitaceae, and Solanaceae, and tobacco, which are recently known to be a host plant for TYLCV, and any plant that is infected with TYLCV may be included therein.

The introduction of the recombinant vector into the plant may be performed using a method known in the art. Examples of the method may include an Agrobacterium-mediated method, particle gun bombardment, silicon carbide whiskers, sonication, heat shock, electroporation, or precipitation using polyethylene glycol (PEG), but are not limited thereto.

Specifically, a method using Agrobacterium tumefaciens may be used to induce TYLCV-specific symptoms in a plant by the TYLCV isolate KG3, KG4, or KG5 of the present disclosure, and the agro-inoculation method may mean a method for introducing and expressing a gene into a plant or producing a desired protein within a plant. Specifically, the agro-inoculation method is that the apex of the plant to be inoculated is wounded using an insect pin and then a suspension of Agrobacterium containing a gene to be transferred is injected into the wounded site.

Accordingly, the present disclosure provides a method for inducing TYLCV symptoms in a plant, the method comprising transfecting the plant with the recombinant vector.

Specifically, the present disclosure provides a method for inducing TYLCV symptoms in a plant, the method comprising transfecting the plant with a recombinant vector, the recombinant vector comprising a nucleic acid molecule comprising the nucleotide sequence of SEQ ID NO: 1, the nucleotide sequence of SEQ ID NO: 2, the nucleotide sequence of SEQ ID NO: 3, or a combination thereof.

More specifically, the present disclosure can obtain a TYLCV-infected plant showing TYLCV-specific symptoms by transfecting a plant with Agrobacterium tumefaciens comprising a recombinant vector comprising a nucleic acid molecule comprising the nucleotide sequence of SEQ ID NO: 1, the nucleotide sequence of SEQ ID NO: 2, the nucleotide sequence of SEQ ID NO: 3, or a combination thereof.

In accordance with still another aspect of the present disclosure, there is provided a composition for diagnosing tomato yellow leaf curl virus (TYLCV) infection, the composition comprising an agent for detecting a nucleic acid molecule comprising the nucleotide sequence of SEQ ID NO: 1, the nucleotide sequence of SEQ ID NO: 2, the nucleotide sequence of SEQ ID NO: 3, or a combination thereof, and a fragment thereof.

Therefore, the composition for diagnosing TYLCV infection of the present disclosure can be used to diagnose the presence or absence of TYLCV infection in a plant.

As used herein, the term “composition” refers to one comprising reagents for DNA detection and amplification, such as primers, DNA polymerases, dNTPs, and buffers for detecting target causative bacteria of symptoms.

As used herein, the term “diagnosis” refers to determining the presence or absence of an infection by amplifying a gene of a subject showing specific symptoms and suspected of inspection, by using the primer set of the present disclosure.

In an embodiment of the present disclosure, the agent for detecting the virus may contain primers or probes specifically binding to TYLCV.

Specifically, the primers or probes specifically binds to the nucleic acid molecule comprising the nucleotide sequence of SEQ ID NO: 1, the nucleotide sequence of SEQ ID NO: 2, the nucleotide sequence of SEQ ID NO: 3, or a combination thereof, and a fragment thereof. More specifically, the primers or probes may be primers or probes set forth in SEQ ID NOS: 20 and 21 below, but are not particularly limited as long as the primers or probes can specifically bind and detect the nucleic acid molecule and the fragment thereof.

The term “specifically binding” refers to a series of nucleotide sequences that synthesize a PCR product by using a gene to be diagnosed as a template only when the gene is present, but cannot synthesize a PCR product when reacting with the other genes.

The composition may further comprise a reaction amplification mixture, and the reaction amplification mixture refers to a solution comprising reagents necessary to perform an amplification reaction, thermostable DNA polymerases, deoxynucleotides, nuclease-free sterile water, and divalent metal cations, and may preferably contain reaction buffers, deoxynucleotides, and DNA polymerases.

An end of the probes may be labelled with a reporter such as a fluorescent substance.

As used herein, the term “primer” refers to a short nucleic acid sequence having a free 3′-hydroxyl group, which can form base pairs with a complementary nucleic acid template and serves as a starting point for strand replication of the nucleic acid template. The primers may initiate DNA synthesis in the presence of reagent for polymerization (that is, DNA polymerase or reverse transcriptase) and four different types of nucleoside triphosphates under appropriate buffer and temperature.

For design of the primers, there are various restrictions, such as the A, G, C, and T content ratios in the primers, prevention of primer dimer formation, and prohibition of repeating the same nucleotide sequence three or more times, and in single PCR reaction conditions, the conditions such as the amount of template DNA, the concentrations of primers, the concentrations of dNTPs, the concentration of Mg²⁺, the reaction temperature, and the reaction time should be appropriate.

The primers may incorporate additional features that do not change the basic properties. That is, the nucleic acid sequence may be modified using many means known in the art. Examples of such modifications include methylation, encapsulation, a substitution of a nucleotide with one or more homologues, and a modification of nucleotides into uncharged linkages, such as phosphonate, phosphotriester, phosphoramidate or carbamate, or charged linkages, such as phosphorothioate or phosphorodithioate. In addition, the nucleic acid may include one or more additional covalently linked residues of nucleases, toxins, antibodies, signal peptides, proteins such as poly L lysine, intercalating agents, such as acridine or psoralen, chelating agents such as metals, radioactive metals, and iron oxidizing metals, and alkylating agents.

The primer sequences of the present disclosure may be modified by using a label that can directly or indirectly provide a detectable signal. The primers may include a label that may be detected using spectroscopic, photochemical, biochemical, immunochemical, or chemical means. Examples of the useful label include proteins for which 32P, fluorescent dyes, electron-dense reagents, enzymes (commonly used in ELISA), biotin or haptens, and antisera or monoclonal antibodies are available.

The primers of the present disclosure may be chemically synthesized by cloning of an appropriate sequence and restriction enzymolysis and a phosphotriester method of Narang et al. (1979, Meth, Enzymol 68:90-99), a diethylphosphoramidite method of Beaucage et al. (1981, Tetrahedron Lett. 22 1859-1862), and any other well-known method including a direct chemical synthesis method such as the solid support method of U.S. Pat. No. 4,458,066.

In accordance with still another aspect of the present disclosure, there is provided a method for diagnosing tomato yellow leaf curl virus (TYLCV) infection in a plant, the method comprising: bringing the composition for diagnosing TYLCV infection into contact with a plant-derived sample, followed by a nucleic acid amplification reaction; and detecting the amplified nucleic acid.

In an embodiment of the present disclosure, the amplified nucleic acid may be selected from the nucleotide sequence of SEQ ID NO: 1, the nucleotide sequence of SEQ ID NO: 2, the nucleotide sequence of SEQ ID NO: 3, or a combination thereof.

Specifically, the method for diagnosing TYLCV infection means a method for detecting TYLCV infection through the identification of the presence or absence of TYLCV in a plant suspected of inspection by using the composition for detecting TYLCV infection to perform an amplification reaction.

More specifically, in an embodiment of the present disclosure, the method of the present disclosure means a method for diagnosing the presence or absence of TYLCV infection by identifying the presence of TYLCV in a plant through the steps of: bringing the composition for diagnosing TYLCV infection into contact with a plant-derived sample suspected of infection, followed by a nucleic acid amplification reaction; and detecting a nucleic acid containing the nucleotide sequence of SEQ ID NO: 1, the nucleotide sequence of SEQ ID NO: 2, the nucleotide sequence of SEQ ID NO: 3, or a combination thereof, or a fragment thereof.

As used herein, the term “amplification reaction” refers to a reaction of amplifying a nucleic acid molecule, which is a polymerase chain reaction (PCR), and examples thereof may include amplification methods, such as reverse transcription polymerase chain reaction (RT-PCR), ligase chain reaction (LCR), Gap-LCR (WO 90/01069), repair chain reaction (EP 439,182), transcription-mediated amplification (TMA) (WO88/10315), self-sustained sequence replication (WO90/06995), selective amplification of target polynucleotide sequences, consensus sequence primed polymerase chain reaction (CP-PCR), arbitrarily primed polymerase chain reaction (AP-PCR), nucleic acid sequence based amplification (NASBA), strand displacement amplification, and loop-mediated isothermal amplification (LAMP), but are not limited thereto.

Since the method for diagnosing TYLCV infection of the present disclosure encompasses the above-described composition for diagnosing TYLCV infection according to another aspect of the present disclosure, the overlapping contents therebetween are recited and the description thereof will be omitted to avoid excessive complexity in the description of the present specification.

The use of the recombinant vector comprising the nucleotide sequence of tomato yellow leaf curl virus (TYLCV) of the present disclosure and Agrobacterium tumefaciens comprising the recombinant vector can effectively induce tomato yellow leaf curl virus infection in a crop even without an insect vector. Furthermore, the infection system of the present disclosure, compared with existing TYLCV Korean isolates, shows excellent infectivity in a plant, and thus can induce symptoms even in TYLCV-resistant varieties of tomatoes having existing widely used Ty-1 or Ty-2 resistant gene, so that the system of the present disclosure can be utilized as an artificial infection system in the future and can be applied in various studies in the future, such as a host plant range for tomato yellow leaf curl virus (TYLCV) and the correlation with the host plant. Furthermore, the infection system of the present disclosure can prevent economical losses due to viral infections in advance and thus can be helpfully used in related fields.

### BRIEF DESCRIPTION OF THE DRAWINGS

FIG. 1 shows a construction procedure of a recombinant plasmid usable as an infectious clone of a novel TYLCV Korean isolate.

FIGS. 2A, 2B, 2C and 2D show the results of TYLCV infectivity by TYLCV-infectious clones in a TYLCV-susceptible variety of tomatoes (Solanum lycopersicum cv. Money Maker).

FIGS. 3A, 3B, 3C and 3D show the TYLCV infectivity by TYLCV-infectious clones in a TYLCV-resistant variety of tomatoes into which TYLCV resistance-related Ty-1 or Ty-2 locus has been introduced.

### DETAILED DESCRIPTION

Hereinafter, the present disclosure will be described in more detail with reference to exemplary embodiments. These exemplary embodiments are provided only for the purpose of illustrating the present disclosure in more detail, and therefore, according to the purpose of the present disclosure, it would be apparent to a person skilled in the art that these exemplary embodiments are not construed to limit the scope of the present disclosure.

### EXAMPLES

Throughout the present specification, the “%” used to express the concentration of a specific material, unless otherwise particularly stated, refers to (wt/wt)% for solid/solid, (wt/vol)% for solid/liquid, and (vol/vol)% for liquid/liquid.

### Example 1: Securing of Novel Tomato Yellow Leaf Curl Virus (TYLCV) Isolates

### Example 1.1. DNA Extraction and Identification of TYLCV Isolates

To secure novel tomato yellow leaf curl virus (TYLCV) isolates first, TYLCV was identified from tomato samples collected from various regions in Korea and genomic DNA thereof was extracted.

Specifically, Danong tomatoes showing typical TYLCV symptoms, such as leaf curling, yellowing, and stunting, were collected from several regions (Chungcheongnam-do, Ganwon-do, Gwangju, Gyeongnam-do, and Jeollanam-do) in Korea, and then viral DNA was extracted from the leaves of a total of 40 collected samples using the Viral Gene-spin™ Viral DNA/RNA Extraction Kit (INTRON Biotechnology, Seongnam, Korea).

Then, the extracted viral DNA was detected using the AccuPower® ProFi Taq PCR Master Mix (Bioneer, Daejeon, Korea) with a final volume of 20 μl containing TYLCV-specific primers encoding the V1 gene of TYLCV isolates, and the T100 Thermal Cycler (Bio-Rad, Hercules, CA, USA) was used for the amplification and DNA detection in PCR. The PCR conditions were as follows: initial denaturation at 94° C. for 3 min, followed by 35 cycles (denaturation at 94° C. for 30 s, annealing at 58° C. for 30 s, and extension at 72ºC for 1 min), and final extension at 72ºC for 10 min. Then, PCR products were electrophoresed on a 1% agarose gel, and thereafter, the PCR products were sequenced using Sanger sequencing at the Macrogen Institute (Macrogen, Seoul, Korea). In this experiment, PCR per DNA sample was performed at least 3 times.

### Example 1.2. Construction of TYLCV Recombinant Plasmids and Securing of Isolates

Newly designed full-genome primers (set forth in SEQ ID NOs: 6 and 7 below, 2.7 K-TYLCV-F and 2.7 K-TYLCV-R) were used to amplify the full sequence of TYLCV from viral DNA of 40 samples confirmed for TYLCV infection in Example 1.1. The target viral DNA was cloned using the pGEM® T-easy vector (Promega, Madison, USA) and then individual recombinant plasmids were sequenced at the Macrogen Institute (Macrogen, Seoul, Korea). After that, the obtained nucleotide sequences were compared for their identities by using the basic local alignment search tool (BLAST) program (blast.ncbi.nlm.nih.gov/Blast.cgi).

A total of 40 TYLCV isolates were secured in this example.

### Example 1.3. Identification of Novel TYLCV Isolates Through Phylogenetic Comparison Analysis

Phylogenetic comparison analysis was performed on full genome sequences of the 40 TYLCV isolate samples obtained in Example 1.2 and the existing TYLCV Korean isolates KG1 (GenBank: HM130912) and KG2 (GenBank: HM130913), and the results confirmed that the 40 TYLCV isolates belong to different clade groups from the existing TYLCV Korean isolates KG1 and KG2, and a total of three novel TYLCV isolates from the 40 isolates were confirmed to newly emerge in Korea.

Specifically, 14 TYLCV isolates separated from a first group were named KG3 (GenBank: ON982178) and showed closest pairwise identity with KG1; a total of 10 TYLCV isolates belong to a second group and were named KG4 (GenBank: ON982198); and 16 TYLCV isolates belonging to the last group were named KG5 (GenBank: ON982202).

### Example 2: Construction of Infectious Clones of Novel TYLCVs

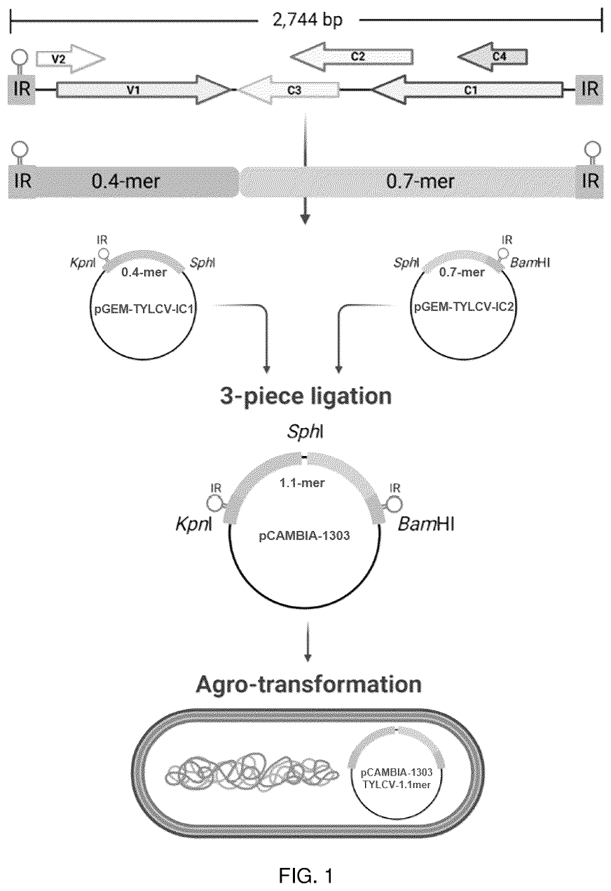

Two fragments of partial tandems were amplified from the full-length recombinant plasmids of novel TYLCV isolates secured in Example 1.3 by using two primer sets for each isolate. The fragments were ligated into the pGEM-T Easy Vector (Promega, Madison, USA) to generate pGEM-TYLCV-IC1 and -IC2, respectively. As shown in FIG. 1, the cloned fragments were digested with the restriction enzymes KpnI, SphI, and BamHI and then cloned into pCAMBIA-1303 digested with the same restriction enzymes. TYLCV-KG3-pCAMBIA1303, TYLCV-KG4-pCAMBIA1303, and TYLCV-KG5-pCAMBIA1303 plasmids thus prepared were treated with restriction enzymes to investigate whether the plasmids were normally constructed. Last, the confirmed plasmids were transformed into Agrobacterium tumefaciens GV3101 strains, respectively, to complete infectious clones.

### Example 3: Identification of Infectivity of Infectious Clones of Novel TYLCV Isolate KG3, KG4, and KG5

First, the TYLCV infectious clones (infectious clones of KG3, KG4, and KG5) prepared in Example 2 were inoculated into tomatoes known as host plants of TYLCV to investigate the infectivity thereof. The infectivity was investigated by measuring the following:
(a) phenotypes of plants;
(b) symptom severity score:
(c) number of viral copies; and
(d) viral detection through PCR analysis.

Specifically, the phenotypes of plants were observed weekly after inoculation for the TYLCV infectious clones, and the symptom severity score was measured from 0 to 4 points according to Friedmann et al. In addition, the genomic DNA was extracted from the TYLCV-inoculated plants by using the FavorPrep Plant Genomic DNA Extraction Mini Kit (Favorgen, Ping-Tung, Taiwan), and then PCR analysis was performed weekly using TYLCV-det-F/R primers to investigate whether TYLCV was present in the infected plants. The number of viral copies was measured by performing qPCR using the Rotor Gene Q thermocycler (QIAGEN, Hilden, Germany).

### Example 3.1. Identification of Infectivity of TYLCV in TYLCV-Susceptible Variety of Tomatoes (Solanum lycopersicum cv. Money Maker)

First, Solanum lycopersicum cv. Money Maker, which is a TYLCV-susceptible variety of tomatoes, was prepared by germination and 4-week cultivation. The prepared tomato plants were infected with novel TYLCV isolates KG3, KG4, and KG5 by treatment with Agrobacterium cultures comprising infectious clones of KG3, infectious clones of KG4, and infectious clones of KG5, respectively. Thereafter, the infectivity was investigated throughout four weeks. It was investigated every week whether the viruses properly infected the plants, by extracting genomic DNA from the leaves of the plants and performing PCR using TYLCV detection primers.

In the present example, groups treated with existing TYLCV isolates KG1 and KG2 were used as positive controls, and a Mock treatment group was used as a negative control. A total of five plants underwent repeated experiments, three times each.

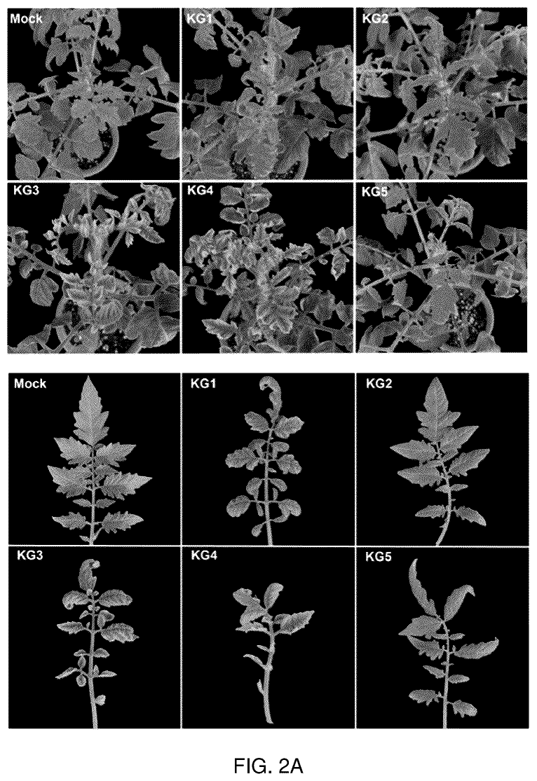

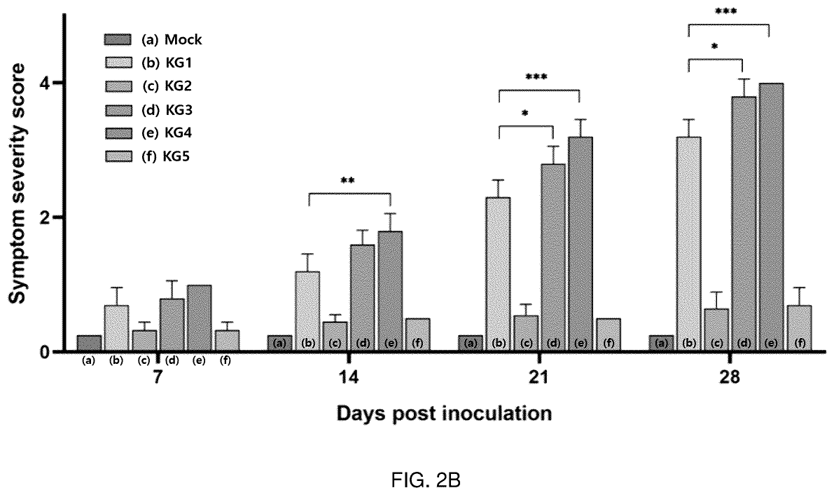

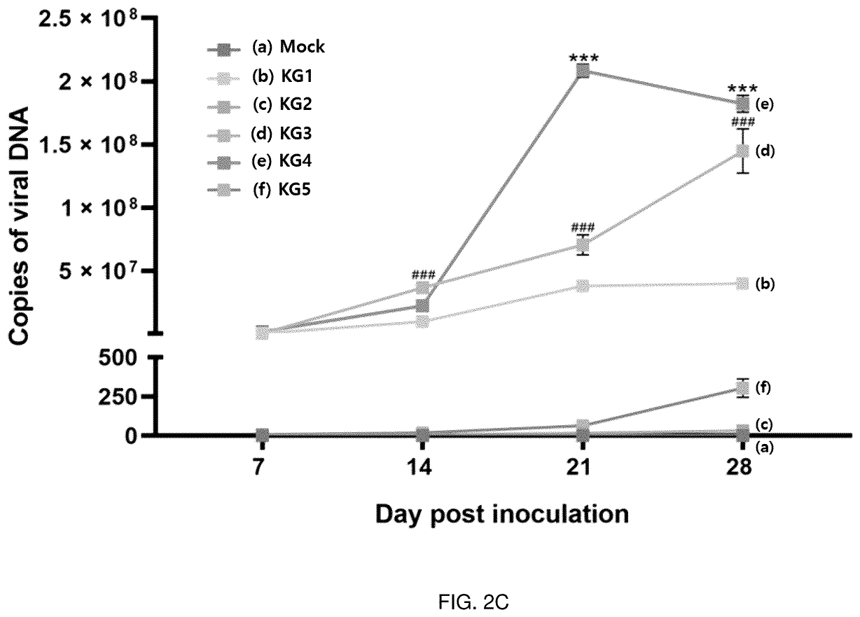

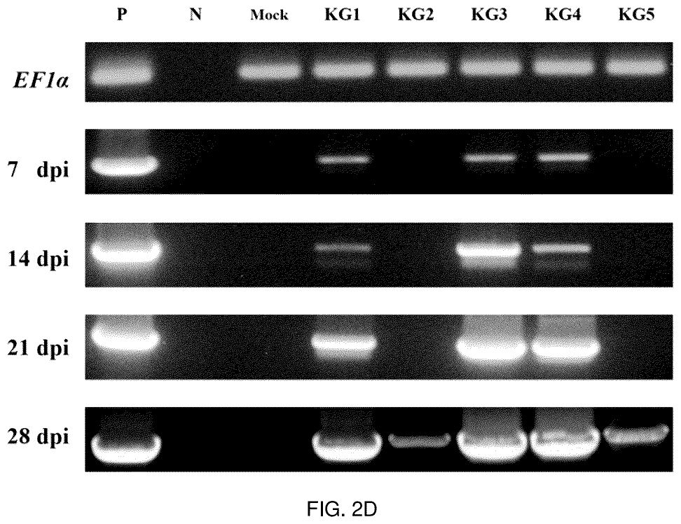

The results are shown in FIG. 2.

As shown in FIG. 2, leaf curling, yellowing, and stunting were observed in all the treatment groups except for the negative control, confirming that TYLCV properly infected the symptom-induced plants.

Specifically, the plants infected with the novel TYLCV isolates KG3 and KG4, compared with KG1 inducing severe symptoms, showed much more severe TYLCV-specific symptoms even when observed with the naked eye and had high symptom severity scores (FIGS. 2A and 2B), and significantly high levels of viral DNA copy numbers. On the other hand, the infection with KG5, compared with KG2-infected plants showing mild symptoms, showed phenotypes of leaf curling and somewhat stunting, and a slightly increased viral DNA copy number at the 4th week of infection (FIGS. 2C and 2D).

It was therefore identified that compared with existing KG1-infected plants showing severe symptoms, KG3 and KG4 could induce much more severe TYLCV-specific symptoms in the plants, and compared with KG2 inducing existing mild symptoms in the infected plants, KG5 was capable of showing more distinctive TYLCV-specific symptoms, confirming that these isolates were novel TYLCV isolates.

### Example 3.2. Identification of TYLCV Infectivity in TYLCV-Resistant Variety of Tomatoes (Ty-1 or Ty-2)

First, a TYLCV-resistant variety of tomatoes with introduced TYLCV-resistance-related Ty-1 or Ty-2 locus were infected with KG1, KG3, and KG4 by treatment with Agrobacterium cultures containing infectious clones of the TYLCV isolate KG1 causing severe symptoms and the novel TYLCV isolates KG3 and KG4, respectively, and after that, the infectivity was investigated. At 7 dpi and 14 dpi, it was investigated whether the viruses properly infected the plants, by extracting genomic DNA from the leaves of the plants and performing PCR using TYLCV detection primers.

In the present example, the TYLCV-susceptible variety of tomatoes (Solanum lycopersicum cv. Money Maker) were used as a positive control and a Mock treatment group was used as a negative control.

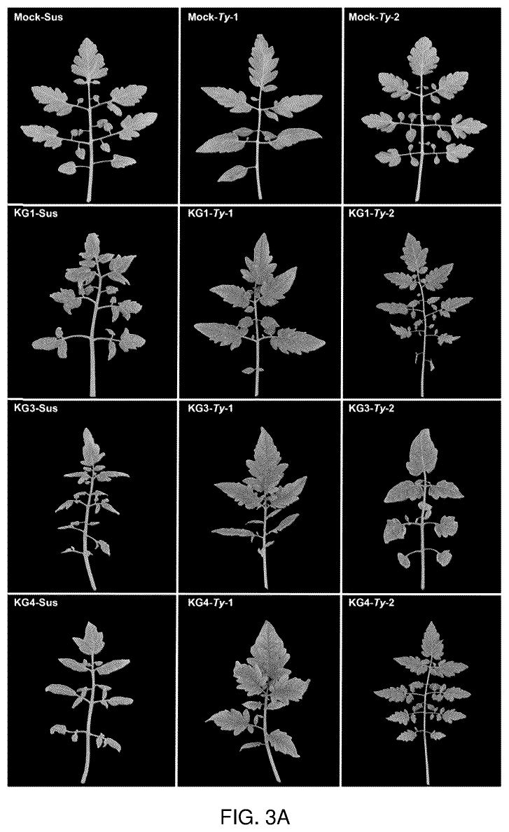

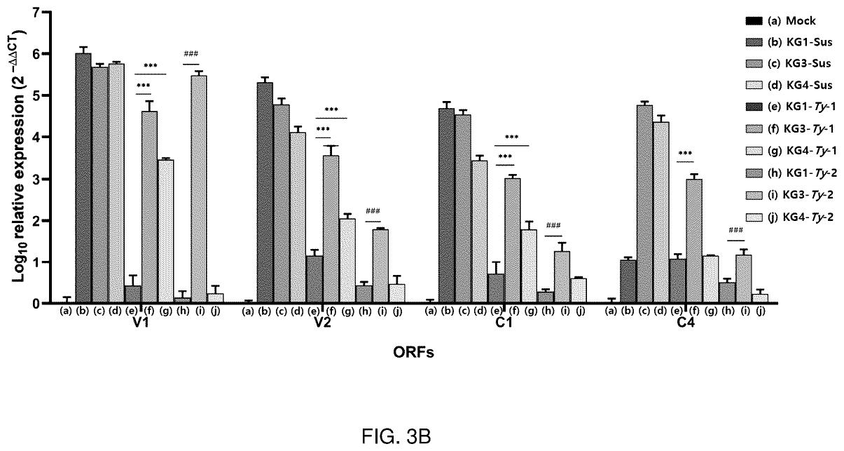

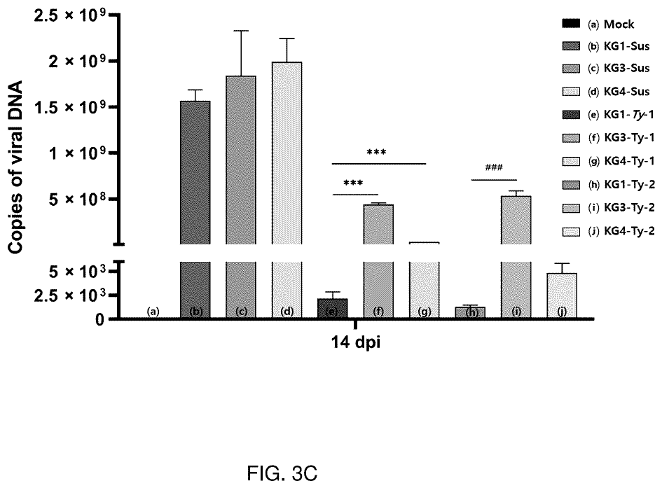

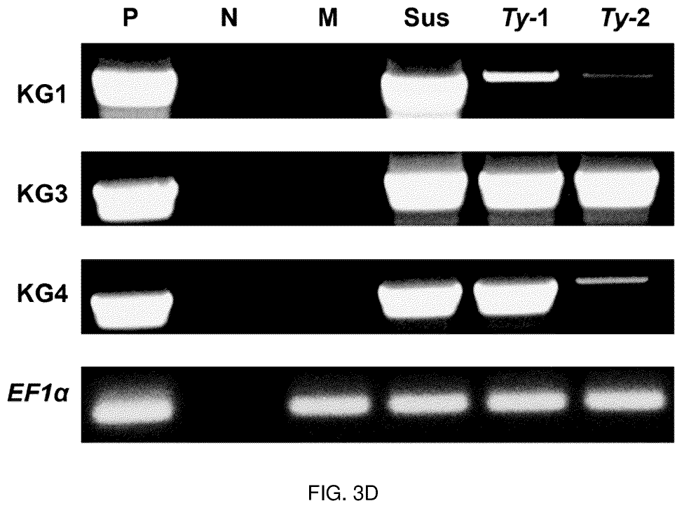

The results are shown in FIG. 3.

Initially, all the TYLCV-resistant variety of tomatoes treated with the TYLCV isolates KG1, KG3, and KG4 were observed to show slight leaf curling, but at 14 dpi, all the TYLCV-resistant variety of tomatoes inoculated with the infectious clones of KG3 continued to show leaf yellowing and curling, but when inoculated with the infectious clones of KG4, only the TYLCV-resistant variety of tomatoes with introduced Ty-1 locus showed symptoms (FIG. 3A). However, compared with the Mock treatment group, all the TYLCV-resistant variety of tomatoes inoculated with the infectious clones of KG1 were not observed to show symptoms.

As shown in FIG. 3B, as a result of investigating the relative expression of 4 ORFs (V1, V2, C1, and C4) for each strain at 14 dpi, the expression of KG3, compared with the KG1-infected plants, was significantly increased in all the TYLCV-resistant variety of tomatoes (P<0.001), and the expression of KG4 showed a significant level in only the TYLCV-resistant variety of tomatoes with introduced Ty-1 locus (P<0.001). The results were identically confirmed in data showing the number of viral DNA copies. Specifically, compared with the plants inoculated with KG1, all the TYLCV-resistant variety of tomatoes inoculated with the infectious clones of KG3 showed a significantly high level of viral copy numbers (P<0.001); on the other hand, KG4 showed significantly high viral DNA copy numbers in only the TYLCV-resistant variety of tomatoes with introduced Ty-1 locus (P<0.001) (FIG. 3C). The infectivity of the novel TYLCV isolates in the TYLCV-resistant variety of tomatoes with introduced Ty-1 or Ty-2 locus was also confirmed by PCR results (FIG. 3D).

It could be therefore identified that the novel TYLCV isolate KG3 showed a high viral DNA copy number and the most severe symptoms even in TYLCV-resistant tomatoes.

What is claimed is:

1. A nucleic acid molecule comprising the nucleotide set forth in SEQ ID NO: 1, the nucleotide set forth in SEQ ID NO: 2, the nucleotide set forth in SEQ ID NO: 3, or a combination thereof.

2. A recombinant vector comprising the nucleic acid molecule of claim 1.

3. Agrobacterium tumefaciens comprising the recombinant vector of claim 2.

4. A tomato yellow leaf curl virus (TYLCV)-infected plant comprising the recombinant vector of claim 2.

5. A composition for detecting tomato yellow leaf curl virus (TYLCV) infection, the composition comprising an agent for detecting the nucleic acid molecule of claim 1 and a fragment thereof.

6. The composition of claim 5, wherein the agent comprises primers or probes specifically binding to the nucleic acid molecule of claim 1 and a fragment thereof.

7. A method for diagnosing tomato yellow leaf curl virus (TYLCV) infection in a plant, the method comprising:
bringing the composition of claim 5 into contact with a plant-derived sample, followed by a nucleic acid amplification reaction; and
detecting the amplified nucleic acid.

8. The method of claim 7, wherein the amplified nucleic acid is a nucleic acid molecule comprising the nucleotide sequence of SEQ ID NO: 1, the nucleotide sequence of SEQ ID NO: 2, the nucleotide sequence of SEQ ID NO: 3, or a combination thereof, or a fragment thereof.

9. A method for inducing tomato yellow leaf curl virus (TYLCV) symptoms in a plant, the method comprising:
transfecting the plant with the recombinant vector of claim 2.


SEQUENCE LISTING (21 sequences)

SEQ ID NO: 1 - DNA, 2774 bp, synthetic construct
  source 1..2774: mol_type = other DNA
accggatggccgcgccttttgcttttatgtggtccccacgagggttacacagacgtcactgtcaaccaatcaaattgcatcctcaaacgttagataagtgttcatttgtctttatatacttggtccccaagttttttgtcttgcaatatgtgggacccacttcttaatgagtttcctgaatctgttcacggatttcgttgtatgttagctattaaatatttgcagtccgttgaggaaacttacgagcccaatacattgggccacgatttaattagggatcttatatctgttgtaagggcccgtgactatgtcgaagcgtccaggcgatataatcatttccacgcccgtctcgaaggttcgccgaaggctgaacttcgacagcccatacagcacccgtgctgctgtccccattgtccaaggcacaaacaagcgacgatcatggacgtacaggcccatgtaccgaaagcccagaatatacagaatgtatcgaagccctgatgttccccgtggatgtgaaggcccatgtaaagtccagtcttatgagcaacgggatgatattaagcatactggtattgttcgttgtgttagtgatgttactcgtggatctggaattactcacagagtgggtaagaggttctgtgttaaatcgatatattttttagggaaagtctggatggatgaaaatatcaagaagcagaatcacactaatcaggtcatgttcttcttggttcgtgatagaaggccctatggaagcagcccaatggattttggacaggtttttaatatgttcgataatgagcccagtaccgcaaccgtgaagaatgatttgcgggataggtttcaagtgatgaggaaatttcatgctacagttattggtggaccctctggaatgaaggaacaggcattagttaagagattttttaaaattaacagtcatgtaacttataatcatcaggaggcagccaagtacgagaaccatactgaaaacgccttgttactgtatatggcatgtacgcatgcctcgaatccagtgtatgcaactatgaaaatacgcatctatttctatgattcaatatcaaattaataaaatttatattttatatcatgagtttctgttacatttattgtgttttcaagtacatcatacaatacatgatcaactgatctgattacattgttaatggaaattacaccaagactatctaaatacttaagaacttcatatctaaatactcttaagaaacgaccagtctgaggctgtaatgtcgtccaaattcggaagttgagaaaacatttgtgaatccccaataccttcctgatgttgtggttgaatcttatctgaatggaaatgatgtcgtggttcattagaaatggcctctggctgtgttctgttatcttgaaatagaggggattgtttatctcccagataaaaacgccattctctgcctgaggagcagtgatgagttcccctgtgcgtgaatccatgattattgcagttgagatggaggtagtatgagcagccacagtctaggtctacacgcttacgccttattggtttcttcttggctatcttgtgttggaccttgattgatacttgcgaacagtggctcgtagagggtgacgaaggttgcattcttgagagcccaatttttcaaggatatgtttttttcttcgtctagatattccttatatgaggaggtaggtcctggattgcagaggaagatagtgggaattccccctttaatttgaatgggcttcccgtactttgtgttgctttgccagtccctttgggcccccataaattccttgaagtgctttaaataatgcgggtctacgtcatcaatgacgttgtaccacgcatcattgctgtacacctttgggcttaggtctagatgtccacataaataattatgtgggcctagagacctggcccacattgttttccctgttctgctatcaccctcaatgacaatacttatgggtctccatggccgcgcagcggaagacacgacgttctcggcgacccactcttcaagttcatctggaacttgattaaaagaagaagaaagaaatggagaaacataaacttctaaaggaggactaaaaatcctatctaaatttgaacttaaattatgaaattgtaaaatatagtcctttggggccttctcttttaatatattgagggcctcggatttactgcctgaattgagtgcttcggcatatgcgtcgttggcagattgctgacctcctctagctgatctgccatcgatttggaaaactccaaaatcaatgaagtctccgtctttctccacgtaggtcttgacatctgttgagctcttagctgcctgaatgttcggatggaaatgtgctgacctgtttggggataccaggtcgaagaaccgttggttcttacattggtatttgccttcgaattggataagcacatggagatgtggttccccattctcgtggagttctctgcaaactttgatgtattttttatttgttggggtttttaggttttttaattgggaaagtgcttcctctttagagagagaacacttgggatatgttaagaaataatttttggcatatatattaaataaacgaggcatgttgaaatgaatcggtgtccctcaaagctctatggcaatcggtgtatcggtgtcttacttatacctggacacctaatggctatttggtaatttcatgaatgttcattgtaattcaaaattcaaaaatcaaatcattaaagcggccatccgtataatatt

SEQ ID NO: 2 - DNA, 2773 bp, synthetic construct
  source 1..2773: mol_type = other DNA
accggatggccgcgccttttgcttttatgtggtccccgcgagggttacacagacgtcactgtcaaccaatcaaattgcatcctcaaacgttagataagtgttcatttgtctttatatacttggtccccaagttttttgtcttgcaatatgtgggacccacttcttaatgagtttcctgaatctgttcacggatttcgttgtatgttagctattaaatatttgcagtccgttgaggaaacttacgagcccaatacattgggccacgatttaattagggatcttatatctgttgtaagggcccgtgactatgtcgaagcggccaggcgatataatcatttccacgcccgtctcgaaggttcgccgaaggctgaacttcgacagcccatacagcacccgtgctgctgtccccattgtccaaggcacaaacaagcgacgatcatggacgtacaggcccatgtaccgaaagcccagaatatacagaatgtatcgaagccctgatgttccccgtggatgtgaaggcccatgtaaagtccagtcttatgagcaacgggatgatattaagcatactggtattgttcgttgtgttagtgatgttactcgtggatctggaattactcacagagttggtaagaggttctgtgttaaatcgatatattttttagggaaagtctggatggatgaaaatatcaagaagcagaatcacactaatcaggtcatgttcttcttagtccgtgatagaaggccctatggaagcagtccaatggattttggacaggtttttaatatgttcgataatgagcccagtaccgcaaccgtgaagaatgatttccgggataggtttcaagtgatgaggaaatttcatgctacagttattggtggaccctctggaatgaaggaacaggccttagttaagagattttttaaaattaacagtcatgtaacttataatcatcaggaggcagccaagtacgagaaccatactgaaaacgccttgttactgtatatggcatgtacgcatgcctcgaatccagtgtatgcaactatgaaaatacgcatctatttctatgattcaatatcaaattaataaaatttatattttatatcatgagtttctgttacatttattgtgttttcaagtacatcatacaatacatgatcaactgatctgattacattgttaatggaaattacaccaagactatctaaatacttaagaacttcatatctaaatactcttaagaaatgaccagtctgaggatgtaatgtcgtccaaattcggaagttgagaaaacatttgtgaatccccatgaccttcctgatgttgtggttgaatcttatctgaatggaaatgatgtcgtggttcattagaaatggcctctggctgtgttctgttatcttgaaatagaggggattgtttatctcccagataaaaacgccattctctgcctgaggagcagtgatgagttcccctgtgcgtgaatccatgattattgcagttgaggtggaggtagtatgagcagccacagtctaggtctacacgcttacgccttattggtttcttcttggctatcttgtgttggaccttgattgatacttgagaacagtggctcgtagagggtgacgaaggttgcattcttgagagtccaatttttcaaggatatgtttttttcttcgtctagatattccctatatgaggaggtaggtcctggattgcagaggaagatagtgggaattccccctttaatttgaatgggcttcccgtactttgtgttgctttgccagtccctttgggcccccataaattccttgaagtgctttaaataatgcgggtctacgtcatcaatgacgttgtaccacgcatcattgctgtacacctttgggcttaggtctagatgtccacataaataattatgtgggcctagacacctggcccacattgttttccctgttctgctatcaccctcaatgacaatacttatgggtctccatggccgcgcagcggaagacacgacgttctcggcgacccactcttcaagttcatctggaacttgattaaaagaagaagaaagaaatggagaaacataaacttctaaaggaggactaaaaatcctatctaaattactgtttaaattatgaaattgtaaaacaaaatctttgggagctttctcccttaatatagacatggccgatgatttagaccctgagttgattgcctcggcatatgcgtcgttggcagactggcaacctcctctagctgatcttccatcgacctggaaaactccaaaatcaacgacgtctccgtctttttccatgtaggtcttgacatctgacgagctcttagctccctgaatgtttggatggaaatgtgctgacctagatggggatacgagatcgaagaatcgttgatttgtgcattgatattttccttcgaattggatgaggacgtggagatgagggctcccatcttcgtgtagttctctgcagattcggatgaataatttatttgttggtgtttctagggcttgaatttgggaaagggcttcttctttggttagagagcagtgtgggtatgtgaggaaataatttttgctatttattttaaaacgctttggtggtgccatatttacaaatatgcccttgtaccccgattcggagagaattcttctttgctgtgtatcggtgtaatggggtacaatatatactccttatgtttttacttttgaattttgaaattcaaaattcaaattggtaaagcggccatccgtataatatt

SEQ ID NO: 3 - DNA, 2791 bp, synthetic construct
  source 1..2791: mol_type = other DNA
accggatggccgcgccttttgcttttatgtggtccccacgagggttacacagacgtcactgtcaaccaatcaaattgcatcctcaaacgttagataactgttcatttgtctttatatacttggtccccaagttttttgtcttgcaatatgtgggacccacttcttaatgagtttcctgaatctgttcacggatttcgttgtatgttagctattaaatatttgcagtccgtcgaggaaacttacgagcccaatacattgggccacgatttaattagggatcttatatctgttgtaagggcccgtgactatgtcgaagcgaccaggcgatataatcatttccacgcccgtctcgaaggttcgccgaaggctgaacttcgacagcccatacagcagccgtgctgctgtccccattgtccaaggcacaaacaagcgacgatcatggacgtacaggcccatgtaccgaaagcccagaatatacagaatgtatcgaagccctgatgttccccgtggatgtgaaggcccatgtaaagtccagtcttatgagcaacgggatgatattaagcatactggtattgttcgttgtgttagtgatgttactcgtggatctggaattactcacagagtgggtaagaggttctgtgttaagtcgatatattttttaggtaaagtctggatggatgaaaatatcaagaagcagaatcacactaatcaggtcatgttcttcttggtccgtgatagaaggccctatggaagcagcccaatggattttggacaggtttttaatatgttcgataacgagcccagtaccgcaaccgtgaagaatgatttgcgtgataggtttcaagtgatgaggaaatttcatgctacagttattggtggaccctctggaatgaaggaacaggcattagttaagagattttttaaaattaacagtcatgtaacttataatcatcaggaggcagccaagtacgagaaccatactgaaaacgccttgttattgtatatggcatgtacgcatgcctcgaatccagtgtatgcaactatgaaaatacgcatttatttctacgattcaatatcaaattaataaaatttatattttatatcatgagtttctgttacatttattgtgttttcaagtacatcatacaatacatgatcaactgatctgattacattgttaatggaaattacaccaaggctatctaaatacttaagaacttgatatctaaatactcttaagaaacgaccagtctgaggatgtaatgtcgtccaaattcggaagttgagaaaacatttgtgaatccccaataccttcctgatgttgtggttgaatcttatttggatggaaatgatgtcgtggttcattagaaatggcctctggctgtgttctgttatcttgaaatagaggggattgtttatctcccagataaaaacgccattctctgcctgaggagcagtgatgagttcccctgtgcgtgaatccatgattattgcagttgaggtggaggtagtatgagcagccacagtctaggtctacacgcttacgccttattggtttcttcttggctatcttgtgttggaccttgattgatacttgcgaacagtggctcgtagagggtgacgaaggttgcattcttgagagcccaatttttcaaggatatgtttttttcttcgtctagatattccctatatgaggaggtaggtcctggattgcagaggaagatagtgggaattccccctttaatttgaatgggcttcccgtactttgtgttgctttgccagtccctttgggcccccataaattccttgaagtgctttaaataatgcgggtctacgtcatcaatgacgttgtaccacgcatcattgctgtacacctttgggcttaggtctagatgtccacataaataattatgtgggcctagagacctggcccacattgttttccctgttctgctatcaccctcaataacaatactattaggtctccatggccgcgcagcggaagacacgacgttctcggatacccatacttcaagttcatctggaacttgattaaaagaagaagaaagaaagggagaaatataaggagccggaggctcttgaaaaattctatctaaattactatttaaattatgaaattgtaaaatataatctttcggtattaattctttaatgattctaagagcctctgacttactgcctgagttaagagctgcggcgtaagcgtcattggctgactgctgacccccacgtgcagatcgtccgtcgatctgaaactcaccccagtcgacggtgtctccgtccttatcgacataagatttgacgtctgaactggatttagctccctgaatgtttggatggaaatgtgctgccctgcttggggataccaggtcgaagaatcgctgattttggcacttgaatttcccttcaaattggataagcacatggagatgtggttccccattctcgtggagctctctgcaaactttgatgtattttttatttgttggagtttctaggttttttaattgggaaagtgcttcttctttcgttaaggagcatttaggatatgtgaggaaataatttttgcaatttatttggaagcgcttagggggagccatatggtcaatgagtaccgattgaccaagatttttacacttatccctggtgtatcggtactcaatatatagtgagtaccaaatggcattttggtaattttataaaagtacattgcaattcaaaattcaaaattcaaaaatcaaatcattaaagcggccatccgtataatatt

SEQ ID NO: 4 - DNA, 2774 bp, synthetic construct
  source 1..2774: mol_type = other DNA
accggatggccgcgccttttccttttatgtggtccccacgagggttacacagacgtcaccgtcaaccaatcaaattgcatcctcaaacgtttgataagtgttcatttgtctttatatacttggtccccaagttttttgtcttgcaatatgtgggacccacttcttaatgagtttcctgaatctgttcacggatttcgttgtatgttagctattaaatatttgcagtccgttgaggaaacttacgagcccaatacattgggccacgatttaattagggatcttatatctgttgtaagggcccgtgactatgtcgaagcgaccaggcgatataatcatttccacgcccgtctcgaaggttcgccgaaggctgaacttcgacagcccatacagcagccgtgctgctgtccccattgtccaaggcacaaacaagcgacgatcatggacgtacaggcccatgtaccgaaagcccagaatatacagaatgtatcgaagccctgatgttccccgtggatgtgaaggcccatgtaaagtccagtcttatgagcaacgggatgatattaagcatactggtattgttcgttgtgttagtgatgttactcgtggatctggaattactcacagagtgggtaagaggttctgtgttaaatcgatatattttttagggaaagtctggatggatgaaaatatcaagaagcagaatcacactaatcaggtcatgttcttcttagtccgtgatagaaggccctatggaagcagcccaatggattttggacaggtttttaatatgttcgataatgagcccagtaccgcaaccgtgaagaatgatttgcgggataggtttcaagtgatgaggaaatttcatgctacagttattggtggaccctctggaatgaaggaacaggcattagttaagagattttttaaaattaacagtcatgtaacttataatcatcaggaggcagccaagtacgagaaccatactgaaaacgccttgttactgtatatggcatgtacgcatgcctcgaatccagtgtatgcaactatgaaaatacgcatctatttctatgattcaatatcaaattaataaaatttatattttatatcatgagtttctgttacatttattgtgttttcaagtacatcatacaatacatgatcaactgatctgattacattgttaatggaaattacaccaagactatctaaatacttaagaacttcatatctaaatactcttaagaaatgaccagtctgaggatgtaatgtcgtccaaattcggaagttgagaaaacatttgtgaatccccatgaccttcctgatgttgtggttgaatcttatctgaatggaaatgatgtcgtggttcattagaaatggcctctggctgtgttctgttatcttgaaatagaggggattgtttatctcccagataaaaacgccattctctgcctgaggagcagtgatgagttcccctgtgcgtgaatccatgattattgcagttgaggtggaggtagtatgagcagccacagtctaggtctacacgcttacgccttattggtttcttcttggctatcttgtgttggaccttgattgatacttgcgaacagtggctcgtagagggtgacgaaggttgcattcttgagagtccaatttttcaaggatatgtttttttcttcgtctagatattccttatatgaggaggtaggtccttgattgcagaggaagatagtgggaattccccctttaatttgaatgggcttcccgtactttgtgttgctttgccagtccctttgggcccccataaattccttgaagtgctttaaataatgcgggtctacgtcatcaatgacgttgtaccacgcatcattgctgtacacctttgggcttaggtctagatgtccacataaataattatgtgggcctagagacctggcccacattgttttccctgttctgctatcaccctcaatgacaatacttatgggtctccatggccgcgcagcggaagacacgacgttctcggcgacccactcttcaagttcatctggaacttgattaaaagaagaagaaagaaatggagaaacataaacttctaaaggaggactaaaaatcctatctaaatttgaacttaaattatgaaattgtaaaatatagtcctttggggccttctcttttaatatattgagggcctcggatttactgcctgaattgagtgcttcggcatatgcgtcgttggcagattgctgacctcctctagctgatctgccatcgatttggaaaactccaaaatcaatgaagtctccgtctttctccacgtaggtcttgacatctgttgagctcttagctgcctgaatgttcggatggaaatgtgctgacctgtttggggataccaggtcgaagaaccgttggttcttacattggtatttgccttcgaattggataagcacatggagatgtggttccccattctcgtggagttctttgcaaactttgatgtattttttatttgttggggtttctaggttttttaattgggaaagtgcttcctctttagagagagaacaattgggatatgttaggaaataatttttggcatatatattaaataaacgaggcatgttgaaatgaatcggtgtccctcaaagctctatggcaatcggtgtatcggtgtcttacttatacctggacacctaatggctatttggtaatttcatgaatgttcattgtaattcaaaattcaaaaatcaaatcattaaagcggccatccgtataatatt

SEQ ID NO: 5 - DNA, 2781 bp, synthetic construct
  source 1..2781: mol_type = other DNA
accggatggacgcgccttttctttttttgtggtccccatgagggttccacagacgtcactgtcaaccaatcaaattgcatcctcaaacgttagataagtgtttatttgtctttatatacttggtccccaagtaatttgtcttgcactatgtgggatccacttctaaatgaatttcctgaatctgttcatggatttcgttgtatgttagctattaaatatttgcaggccgttgaggaaacttacgagcccaatacattgggccacgatttaattagggatcttatatctgttgtaagggcccgtgactatgtcgaagcgaccaggcgatataatcatttccacgcccgtctcgaaggttcgccgaaggctgaacttcgacagcccatacagcagccgtgctgctgtccccattgtccaaggcacaaacaagcgacgatcatggacgtacaggcccatgtaccggaagcccagaatatacagaatgtatcgaagccctgatgttcctcgtggatgtgaaggcccatgtaaagtccagtcttatgagcaacgggatgatattaagcatactggtattgttcgttgtgttagtgatgttactcgtggatctggaattacccacagagtgggtaagaggttctgtgttaaatcgatatattttttaggtaaagtctggatggatgaaaatattaagaagcagaatcacactaatcaggtcatgttcttcttggttcgtgatagaaggccctatggaaacagcccaatggattttggacaggtttttaatatgttcgataatgagcccagtaccgcaaccgtgaagaatgatttgcgggataggtttcaagtgatgaggaaatttcatgctacagttattggtgggccctctggaatgaaggaacaggcattagttaagagattttttagaattaacagtcacgtaacttataatcatcaggaggcagccaagtatgagaaccatactgaaaacgccttgttattgtatatggcatgtacgcatgcctctaatccagtgtatgcaactatgaaaatacgcatctatttctatgattcaatatcaaattaataaaatttatattttatatcatgagtttctgttacatttattgtgttttcaagtacatcatacaatacatgatcaactgctctgattacattattaattgaaattacaccaagactatctaaatacttaagaatttgatatctaaatactcttaataaacgaccagtctgaggctgtaatgtcgtccaaattcggaagttgagaaaacatttgtgaatccccaataccttcctgatgttgtggttgaatcttatctgaatggaaatgatgtcgtggttcattagaaatggcctctggctgtgttctgttatcttgaaatagaggggattgtttatctcccagataaaaacgccattctctgcttgaggagcagtgatgagttcccctgtgcgtgaatccatgattgttgcagttgatgtggaggtagtatgagcagccacagtctaggtctacacgcttacgccttatgggtttcttcttggctatcttgtgttggaccttgattgatacttgtgaacagtggctcgtagagggtgacgaaggttgcattcttgagagcccaatttttcaatgatatgtttttttcttcgtctagatattccctatatgaggaggtaggtcctggattgcagaggaagatagtgggaattccccctttaatttgaatgggcttcccgtactttgtgttgctttgccagtccctctgggcccccatgaattccttgaagtgctttaaataatgcgggtctacgtcatcaatgacgttgtaccacgcatcattactgtacacctttgggcttaggtctagatgtccacataaataattatgtgggcctagagacctggcccacattgttttgcctgttctgctatcaccctcaatgacaatacttatgggtctccatggccgcgcagcggaagatatgacgttctcggcgacccactcttcaagttcatctggaacttgattaaaagaagaagaaagaaatggagaaacataaacttctaaaggaggactaaaaatcctatctaaatttgaacttaaattatgaaattgtaaaatatagtcctttggggccttctcttttaatatattgagggcctcggatttactgcctgaattgagtgcctcggcatatgcgtcgttggcagattgctgacctcctctagctgatctgccatcgatttggaaaactccaaaatcaatgaagtctccgtctttttccacgtaggtcttgacatctgttgagctcttagctgcctgaatgttcggatggaaatgtgctgacctgtttggggataccaggtcgaagaaccgttggttcttacattggtatttgccttcgaattggataagcacatgtagatgtggttccccattctcgtggagttctctgcaaactttgatgtattttttatttgttggggtttctaggtttgttaattgggaaagggcttcctctttagagagagaacaattgggatatgttaggaaataatttttggcatatattttaaataaacgaggcatgttgaaatgaattggtgtccctcaaagctctatggcaatcggtgtatcggtgtcttacttatacctggacacctaatggctatttggtaattttgtaaaagtacaattcaattcaaaattcaaaattcaaaaatcaaatcattaaagcggccatccgtataatatt

SEQ ID NO: 6 - DNA, 29 bp, synthetic construct
  source 1..29: mol_type = other DNA
gatattaagcatactggtattgttcgttg

SEQ ID NO: 7 - DNA, 21 bp, synthetic construct
  source 1..21: mol_type = other DNA
tcatcccgttgctcataagac

SEQ ID NO: 8 - DNA, 27 bp, synthetic construct
  source 1..27: mol_type = other DNA
ggtaccgttgaaatgaatcggtgtccc

SEQ ID NO: 9 - DNA, 31 bp, synthetic construct
  source 1..31: mol_type = other DNA
agaggcatgcgtacatgccatatacagtaac

SEQ ID NO: 10 - DNA, 31 bp, synthetic construct
  source 1..31: mol_type = other DNA
gtacgcatgcctctaatccagtgtatgcaac

SEQ ID NO: 11 - DNA, 29 bp, synthetic construct
  source 1..29: mol_type = other DNA
ggatccattgcaagacaaaaaacttgggg

SEQ ID NO: 12 - DNA, 29 bp, synthetic construct
  source 1..29: mol_type = other DNA
ggtaccatttacaaatatgcccttgtacc

SEQ ID NO: 13 - DNA, 31 bp, synthetic construct
  source 1..31: mol_type = other DNA
cgaggcatgcgtacatgccatatacagtaac

SEQ ID NO: 14 - DNA, 31 bp, synthetic construct
  source 1..31: mol_type = other DNA
gtacgcatgcctcgaatccagtgtatgcaac

SEQ ID NO: 15 - DNA, 29 bp, synthetic construct
  source 1..29: mol_type = other DNA
ggatccattgcaagacaaaaaacttgggg

SEQ ID NO: 16 - DNA, 29 bp, synthetic construct
  source 1..29: mol_type = other DNA
ggtaccatggtcaatgagtaccgattgac

SEQ ID NO: 17 - DNA, 31 bp, synthetic construct
  source 1..31: mol_type = other DNA
cgaggcatgcgtacatgccatatacaataac

SEQ ID NO: 18 - DNA, 31 bp, synthetic construct; identical to SEQ ID NO: 14
  source 1..31: mol_type = other DNA

SEQ ID NO: 19 - DNA, 29 bp, synthetic construct
  source 1..29: mol_type = other DNA
ggatccattgcaagacaaaaaacttgggg

SEQ ID NO: 20 - DNA, 21 bp, synthetic construct
  source 1..21: mol_type = other DNA
aagcgaccaggcgatataatc

SEQ ID NO: 21 - DNA, 20 bp, synthetic construct
  source 1..20: mol_type = other DNA
aggggaactcatcactgctc